(19) United States
(12) Patent Application Publication
(10) Pub. No.: US 2006/0167259 A1
(43) Pub. Date: Jul. 27, 2006

(54) Direct racemization of indole derivatives

(75) Inventors: Qi Chao, San Diego, CA (US); Jian Chen, Malvern, PA (US)

Correspondence Address: CEPHALON, INC., 41 MOORES ROAD, PO BOX 4011, FRAZER, PA, 19355, US

(73) Assignee: Cephalon, Inc., Frazer, PA (US)

(21) Appl. No.: 11/335,406

(22) Filed: Jan. 19, 2006

Related U.S. Application Data
(60) Provisional application No. 60/645,867, filed on Jan. 21, 2005.

(51) Int. Cl. C07D 498/02; C07D 491/02; C07D 491/052; C07D 471/02

(52) U.S. Cl. 546/85; 548/432

(57) ABSTRACT

The present invention discloses processes for the racemization of enantiomers of etodolac and other tetra-hydropyrano indole derivatives.

### FIELD OF THE INVENTION

The present invention concerns reactions useful for racemizing enantiomers of indole derivatives, such as (R)-etodolac or (S)-etodolac.

### BACKGROUND

The following description includes information that may be useful in understanding the present invention. It is not an admission that any such information is prior art, or relevant, to the presently claimed inventions, or that any publication specifically or implicitly referenced is prior art. 1,8-Diethyl-1,3,4,9-tetrahydropyrano[3,4-b]indole-1-acetic acid, also known as etodolac, is a non-steroidal analgesic anti-inflammatory agent. Etodolac is a chiral compound (indicated by the asterisk in generic Formula I below). Due to its chiral center, this drug exists as an enantiomeric mixture of R- and S-etodolac. Demerson et al. reported that etodolac's anti-inflammatory and analgesic properties reside in only one of its enantiomers, the S form (1983 J Med Chem 26:1778).

Etodolac also kills cancer cells. Both enantiomers of etodolac kill cancer cells, but since the S-enantiomer contains undesirable side effects, R-etodolac is preferred for treatment of cancers. The use of etodolac and etodolac analogs to treat cancer and other conditions is disclosed in U.S. Pat. Nos. 6,573,292; 6,545,034; 5,955,504, and International Publication Nos. WO 02/12188; WO 01/06990; and WO 2004/026116.

The preparation of etodolac is described in U.S. Pat. No. 3,843,681. Processes for producing a single enantiomer of etodolac are disclosed in U.S. Pat. Nos. 4,520,203; 4,515,961; and 5,811,558.

U.S. Pat. No. 3,843,681 also describes compounds that are analogs of etodolac and all within the following formula (Formula I): 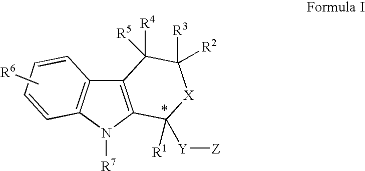
in which R¹ is H, a lower alkyl, lower alkenyl, lower cycloalkyl, phenyl, benzyl, or thienyl; R², R³, R⁴, and R⁵, which may be the same or different from each other, are H or a lower alkyl; R⁶ is a lower alkyl, lower cycloalkyl, hydroxy, alkoxy, benzyloxy, alkanoyloxy, phenyl, nitro, halogen, mercapto, alkylthio, trifluoromethyl, amino, or sulphamoyl; R⁷ is H, a lower alkyl, or lower alkenyl; X is O or S; Y is —CO—, —CR⁸(R⁹)—CR¹⁰(R¹¹)—CO—, —CR⁸(R⁹)CR¹⁰(R¹¹)CR¹² (R¹³)—CO—, in which R⁸, R⁹, R¹⁰, R¹¹, R¹² and R¹³, which are the same or different from each other, are H or a lower alkyl; Z is OH, a lower alkoxy, a lower alkylamino, or a lower dialkylamino.

Other related indole compounds have also been shown to exhibit similar anti-inflammatory activity as etodolac. For example, U.S. Pat. Nos. 3,843,681; 3,939,178; 3,974,179; 4,070,371; 4,686,213; 4,748,252; and International Publication No. WO 02/12188 disclose indole derivatives based on the 1,3,4,9-tetra-hydropyrano[3,4-b]-indole-1-acetic acid nucleus that are stated to exhibit anti-inflammatory, analgesic, antibacterial and/or antifungal activity. Similar 1,2,3,4-tetrahydro-4H-carbazole and 2,3,4,9-1H-carbazole compounds and their use as cyclooxygenase-2 (COX-2) inhibitors for antiarthritic, colorectal cancer and Alzheimer's therapy are also disclosed in U.S. Pat. Nos. 5,776,967; 5,824,699; and 5,830,911.

U.S. Pat. No. 5,811,558 discloses that enantiomeric esters of etodolac can be racemized by a catalytic amount of acid, acid resin, or base.

Mizuguchi et al. reported that treatment of optically active (R)-etodolac by SOCl₂ in ethanol gave the corresponding racemic ethyl ester (Heterocycles, 1997, 46:149).

During the production of single enantiomers of etodolac during the process of chemical synthesis or resolution of the racemic mixture, these processes will produce both the R and S forms. Thus, the form not used may end up being discarded. Further, the racemization method disclosed in U.S. Pat. No. 5,811,558 requires that esters of etodolac be used as the starting materials. Additionally, the racemization process disclosed by Mizuguchi et al., although starting with etodolac, ends with a product that is an ester. Therefore, a need exists for the efficient direct racemization of a single enantiomer of etodolac or a tetra-hydropyrano indole derivative that allows regeneration of the racemic mixture for future use in producing separate enantiomers. Direct racemization of enantiomers of etodolac and tetra-hydropyrano indole derivatives is advantageous in terms of chemical and economical efficacy.

### SUMMARY OF THE INVENTION

The present invention involves a process for the racemization of either the R-enantiomer or S-enantiomer of compounds of Formula II: 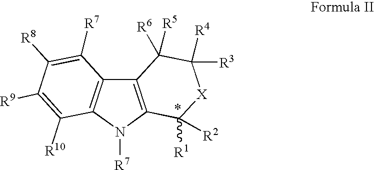
wherein R¹, R², R³, R⁴, R⁵, R⁶, R⁷, R⁸, R⁹, R¹⁰ and R¹¹ are each independently hydrogen; halogen; —CN; —NO₂; —OH; —SH; or an unsubstituted or substituted moiety selected from alkyl, alkenyl, alkynyl, alkoxy, allyloxy, aryl, heteroaryl, cycloalkyl, and heterocycloalkyl; X is O, N, or S; where R¹ and R² are not the same and R¹ and R² are not an ester; and where the process includes:
a) adding a compound of Formula II to a reaction solvent; and
b) adding a Lewis acid and/or Brønsted acid to the reaction mixture; provided that when the acid is H₂SO₄, HCl, or para-toluenesulfonic acid the reaction solvent is not methyl alcohol.

A preferred compound for the racemization reactions disclosed herein is a single enantiomer of etodolac.

Other preferred compounds for the racemization reactions disclosed herein include the following:

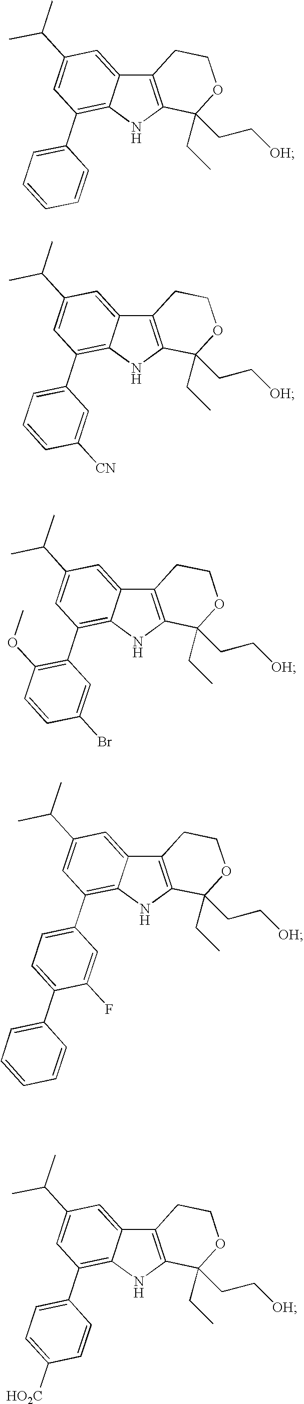

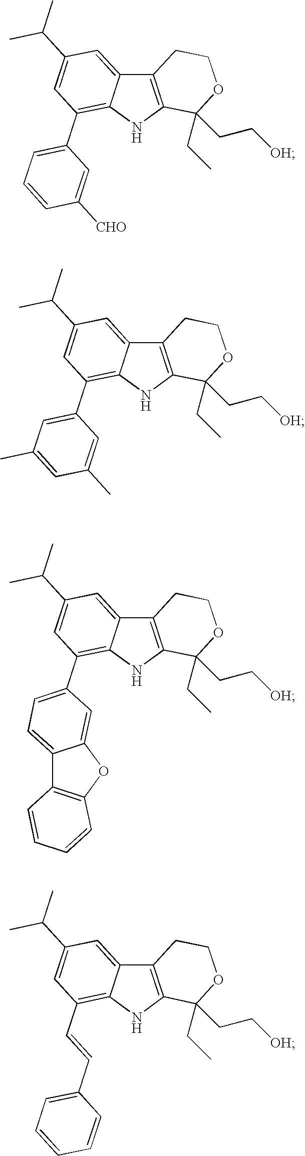

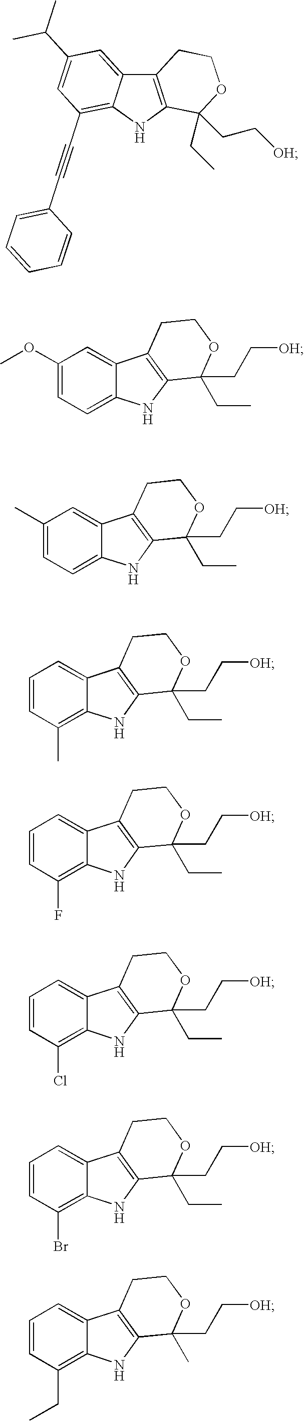

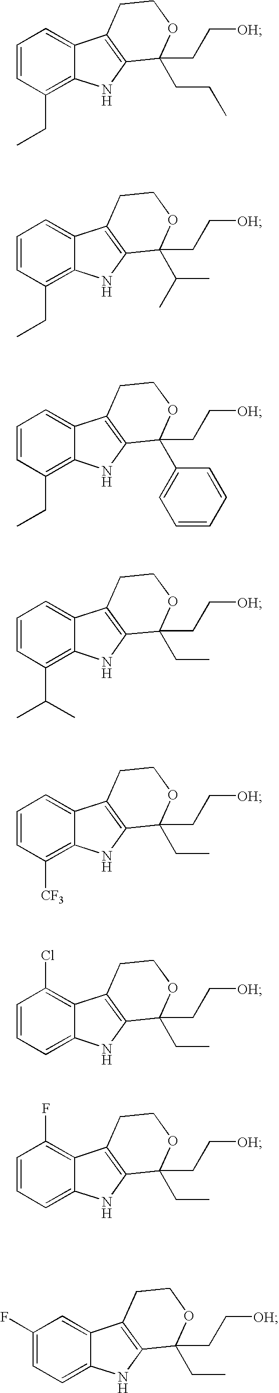

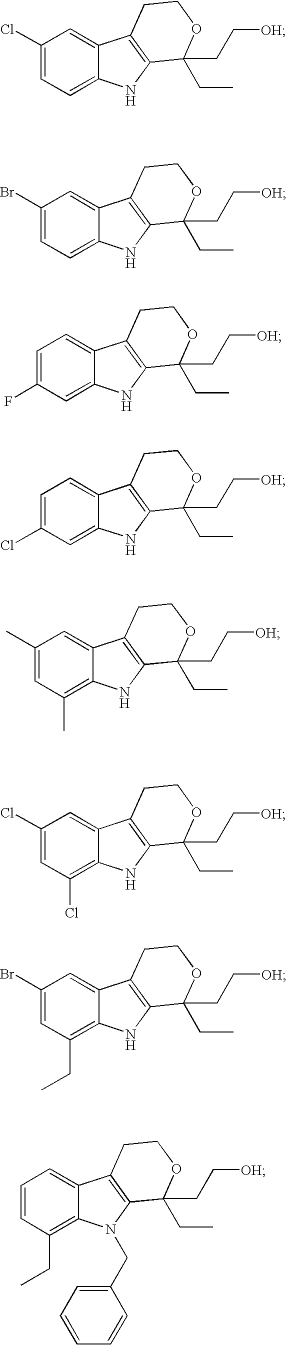

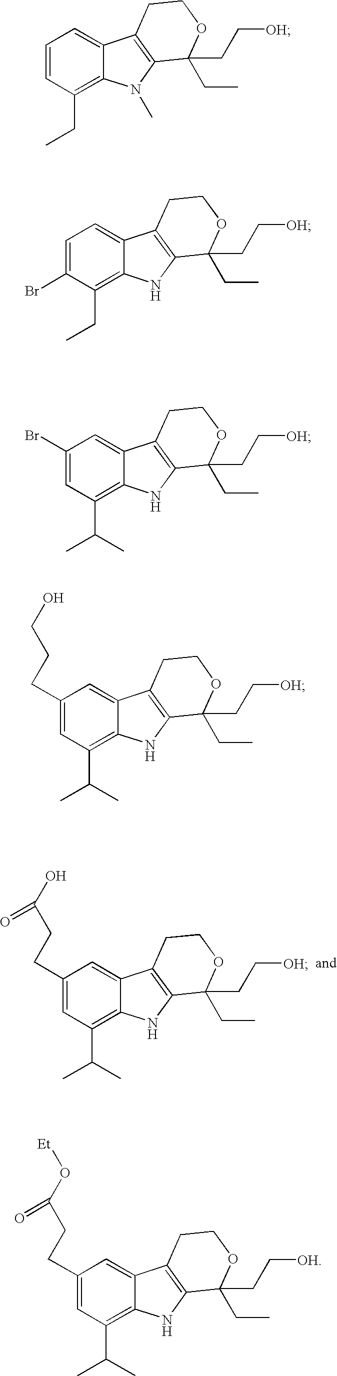

To purify the reaction product, the processes disclosed herein can include purification steps which involve removing the reaction solvent and the Lewis acid and/or Brønsted acid. Those of reasonable skill in the art understand that this can be accomplished using a number of methods. Persons of reasonable skill in the art also understand that although the racemization process and purification process are shown in a certain order of steps, in some instances, the order of the steps may be changed. Examples of preferred purifications steps include the following: removing the reaction solvent; adding water after the removal of the reaction solvent; neutralizing the aqueous mixture to pH 4-5; extracting with an organic liquid such as dichloromethane; drying the organic phases over MgSO₄; filtering the organic phases; concentrating the organic phases; and drying the resultant residue in vacuo. A more preferred purification procedure involves the following: removing the reaction solvent under reduced pressure; adding a liquid (preferably water) to the resulting residue; neutralizing the mixture to about pH 10; extracting with ethyl acetate; acidifying the aqueous phase to about pH 4-5; extracting with dichloromethane; drying the organic phases over MgSO₄; filtering and concentrating the dried phases; and drying in vacuo.

Reaction solvents for the process described herein can include, without limitation, one or more of a lower alcohol, CH₃CN, 1,4-dioxane, or CH₂Cl₂. Preferred reaction solvents include methyl alcohol, 2-propanol, ethyl alcohol, 2-methyl propan-1-ol, butan-2-ol and tert-butyl alcohol. More preferred reaction solvents include 2-propanol and ethyl alcohol.

Direct racemization of a single enantiomer of a compound of Formula II occurs with the addition of a Lewis acid and/or Brønsted acid to an organic solvent containing a compound of Formula II. Preferred acids include, without limitation, one or more of SOCl₂, SnC₄, TiCl₄, AlCl₃, La(CF₃SO₃)₃, BF₃-Et₂O and H₂SO₄. More preferred acids are BF₃-Et₂O and H₂SO₄. Preferred concentrations of an acid for the racemization process are about 0.75 to about 1.5 equivalents. A more preferred concentration is about 1.5 equivalents.

Although the processes disclosed herein are not limited as to time or temperature, preferred reaction times are greater than about 30 minutes to about 36 hours. More preferred reactions times are about 4 to 20 hours. Even more preferred reactions times are about 18 hours. With respect to temperature, reactions are typically run at temperatures that are below 50° C., more preferably the reaction is carried out at about 18° C. to about 26° C. A preferred temperature for the reaction is about 20° C.

### DETAILED DESCRIPTION OF THE INVENTION

A. Definitions

As used herein and in the appended claims, the singular forms “a”, “an”, and “the” include plural reference unless the context clearly dictates otherwise.

In accordance with a convention used in the art, [symbol] is used in structural formulae herein to depict the bond that is the point of attachment of the moiety or substituent to the core or backbone structure.

In accordance with a convention used in the art, the symbol [symbol] represents a methyl group, [symbol] represents an ethyl group, [symbol] represents a cyclopentyl group, etc.

The term “alkyl” as used herein refers to a straight- or branched-chain alkyl group having one to twelve carbon atoms. Exemplary alkyl groups include methyl (Me), ethyl (Et), n-propyl, isopropyl, butyl, isobutyl, sec-butyl, tert-butyl (tBu), pentyl, isopentyl, tert-pentyl, hexyl, isohexyl, and the like. The term “lower alkyl” designates an alkyl having from 1 to 6 carbon atoms (a C₁₋₆-alkyl).

The term “heteroalkyl” as used herein refers to straight- and branched-chain alkyl groups having from one to twelve atoms containing one or more heteroatoms selected from S, O, and N. The term “lower heteroalkyl” designates a heteroalkyl having from 1 to 6 carbon atoms (a C₁₋₆-heteroalkyl).

The term “haloalkyl” as used herein refers to straight- and branched-chain alkyl groups having from one to four carbon atoms wherein one or more carbon atoms is substituted with one or more halogen atoms. An example of haloalkyl is —CF₃.

The term “alkenyl” means an alkyl radical having one or more double bonds and two to twelve carbon atoms. Alkenyl groups containing three or more carbon atoms may be straight or branched. Alkenyl groups as used herein include either the cis or trans configurations. Illustrative alkenyl groups include prop-2-enyl, but-2-enyl, but-3-enyl, 2-methylprop-2-enyl, hex-2-enyl, and the like. The term “lower alkenyl” designates an alkenyl having from 2 to 6 carbon atoms (a C₂₋₆-alkenyl).

The term “allyloxy” refers to an alkenyloxy group which is CH₂═CHCH₂—O—.

The term “alkynyl” means an alkyl radical having one or more triple bonds and two to twelve carbon atoms. Alkynyl groups containing four or more carbon atoms may be straight or branched. Alkynyl groups as used herein include either the cis or trans configurations. Illustrative alkynyl groups include prop-2-ynyl, but-2-ynyl, but-3-ynyl, 2-methylbut-2-ynyl, hex-2-ynyl, and the like. The term “lower alkynyl” designates an alkynyl having from 2 to 6 carbon atoms (a C₂₋₆-alkynyl).

The term “aryl” (Ar) refers to a monocyclic, or fused or spiro polycyclic, aromatic carbocycle (ring structure having ring atoms that are all carbon) having from six to fourteen ring atoms per aryl moiety. Illustrative examples of aryl groups include the following moieties: 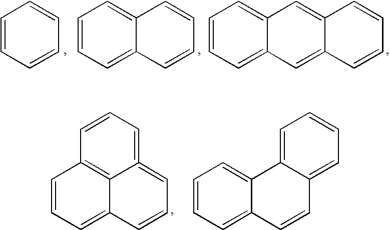
, and the like.

The term “heteroaryl” (heteroAr) refers to a monocyclic, or fused or spiro polycyclic, aromatic heterocycle (ring structure having ring atoms selected from carbon atoms as well as one, two, or three heteroatoms selected from nitrogen, oxygen, and sulfur) having from five to fourteen ring atoms per heteroaryl moiety. Illustrative examples of heteroaryl groups include the following moieties: 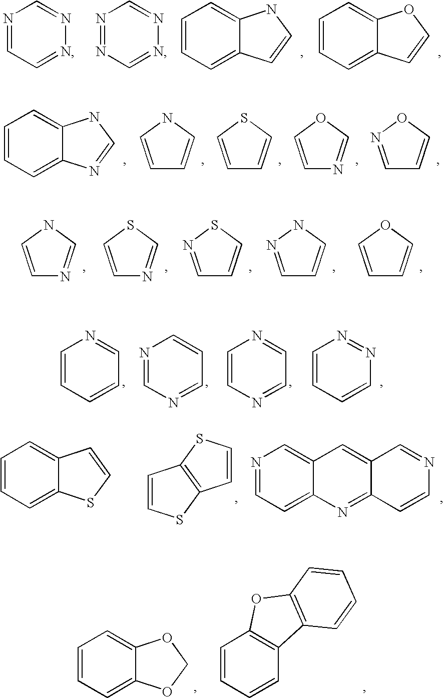
and the like.

The term “cycloalkyl” refers to a saturated or partially saturated, monocyclic or fused or spiro polycyclic, carbocycle having from three to twelve ring atoms per cycloalkyl moiety. Illustrative examples of cycloalkyl groups include the following moieties: 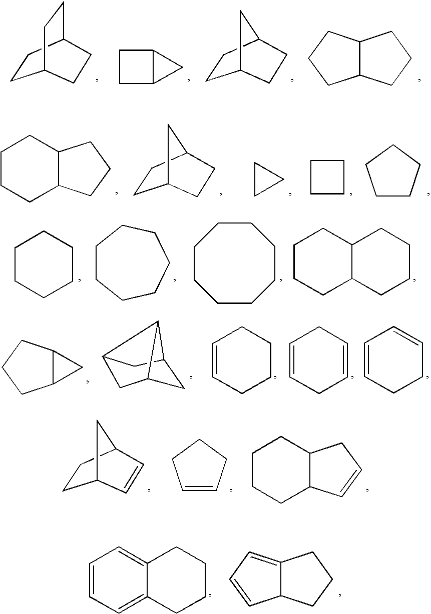
and the like.

A “heterocycloalkyl” refers to a monocyclic, or fused or spiro polycyclic, ring structure that is saturated or partially saturated and has from three to twelve ring atoms per heterocycloalkyl moiety and the ring structure having ring atoms selected from carbon atoms as well as one, two, three or four heteroatoms selected from nitrogen, oxygen, and sulfur. Illustrative examples of heterocycloalkyl groups include: 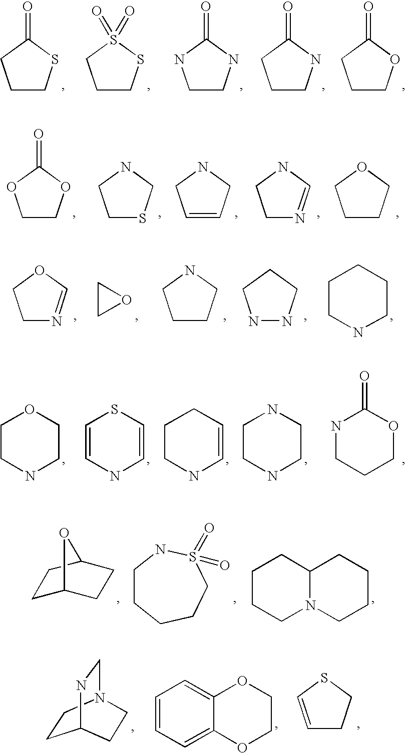
and the like.

The term “alkoxy” refers to alkyl-O—. Illustrative examples include methoxy, ethoxy, propoxy, and the like.

The term “halogen” represents chlorine, fluorine, bromine or iodine. The term “halo” represents chloro, fluoro, bromo or iodo.

The term “lower” when referring to a group such as an alkyl, alkenyl, alkynyl, alkoxy or other group refers to such a group having up to 6 carbon atoms.

The term “substituted” as used herein means any of the above groups (e.g., alkyl, alkenyl, alkynyl, alkoxy, allyoxy, aryl, arylalkyl, heteroaryl, cycloalkyl and heterocycloalkyl) wherein at least one hydrogen atom is replaced with a substituent. In the case of an oxo substituent (═O) two hydrogen atoms are replaced. When one or more of the above groups are substituted, “substituents” within the context of this invention include ═O, ═S, —CN, —NO₂, alkyl, alkenyl, heteroalkyl, haloalkyl, alkynyl, aryl, cycloalkyl, heterocycloalkyl, heteroaryl, —(CH₂)_{z}CN where z is an integer from 1 to 4, ═NH, —NHOH, —OH, —C(═O)H, —OC(═O)H, —C(═O)OH, —OC(═O)OH, —OC(═O)OC(═O)H, —OOH, —C(═NH)NH₂, —NHC(═NH)NH₂, —C(═S)NH₂, —NHC(═S)NH₂, —NHC(═O)NH₂, —S(O₂)H, —S(═O)H, —NH₂, —C(═O)NH₂, —OC(═O)NH₂, —NHC(═O)H, —NHC(═O)OH, —C(═O)NHC(═O)H, —OS(O₂)H, —OS(═O)H, —OSH, —SC(═O)H, —S(═O)C(═O)OH, —SO₂C(═O)OH, —NHSH, —NHS(═O)H, —NHSO₂H, —C(═O)SH, —C(═O)S(═O)H, —C(═O)S(O₂)H, —C(═S)H, —C(═S)OH, —C(SO)OH, —C(SO₂)OH, —NHC(═S)H, —OC(═S)H, —OC(═S)OH, —OC(SO₂)H, —S(O₂)NH₂, —S(═O)NH₂, —SNH₂, —NHCS(O₂)H, —NHC(SO)H, —NHC(═S)H, and —SH groups. In addition, the above substituents may be further substituted with one or more substituents independently selected from the group consisting of halogens, ═O, —NO₂, —CN, —(CH₂)_{z}—CN where z is an integer from 1 to 4, —OR^{c}, —NR^{c}OR^{c}, —NR^{c}R^{c}, —C(═O)NR^{c}, —C(═O)OR^{c}, —C(═O)R^{c}, —NR^{c}C(═O)NR^{c}R^{c}, —NR^{c}C(═O)R^{c}, —OC(═O)OR^{c}, —OC(═O)NR^{c}R^{c}, —SR^{c}, unsubstituted alkyl, unsubstituted alkenyl, unsubstituted alkynyl, unsubstituted aryl, unsubstituted cycloalkyl, unsubstituted heterocycloalkyl, and unsubstituted heteroaryl, or two or more substituents cyclize to form a fused or spiro polycyclic cycloalkyl, heterocycloalkyl, aryl, or heteroaryl group, where R^{c} is hydrogen, unsubstituted alkyl, unsubstituted alkenyl, unsubstituted alkynyl, unsubstituted aryl, unsubstituted cycloalkyl, unsubstituted heterocycloalkyl, or unsubstituted heteroaryl, or two or more R^{c} groups together cyclize to form part of a heteroaryl or heterocycloalkyl group unsubstituted or substituted with an unsubstituted alkyl group.

The term “unsubstituted” means that the specified group bears no substituents.

The term “acid” as used herein, means an organic or inorganic acid. Representative examples of organic acid include, but are not limited to oxalic acid, tartaric acid, acetic acid, formic acid, trifluoroacetic acid and p-toluenesulfonic acid. Representative examples of inorganic acids include, but are not limited to, hydrochloric acid (HCl) and hydrobromic acid (HBr).

The term “Lewis acid” as used herein, means a chemical species, other than a proton, that has a vacant orbital or accepts an electron pair. It is to be understood that Lewis acids can be purchased or prepared as complexes including but not limited to, etherates, hydrates, and thioetherates. Representative examples of Lewis acids include, but are not limited to, aluminum chloride, bismuth (III) chloride, boron trifluoride, boron trifluoride etherate, iron (II) chloride, iron (III) chloride, lanthanide triflates, magnesium bromide, magnesium chloride, magnesium trifluoromethanesulfonate, manganese (II) chloride, thionyl chloride, tin (IV) chloride, titanium tetra chloride, zinc bromide, zinc chloride, zirconium (IV) chloride, lanthanum triflates, such as La(CF₃SO₃)₃ and the like. In general, the Lewis acids that can be used include triflates and halides of elements of groups IB, IIB, IIIB, IVB, VB, VIB, VIIB, VIII, IIIA, IVA, lanthanides, and actinides (American Chemical Society format).

As used herein a “Brønsted acid” is a molecular entity capable of donating a hydrogen (proton) to a base, (i.e., a ‘hydrogen donor’) or the corresponding chemical species. For example, H₂O, H₃O⁺, CH₃CO₂H, H₂SO₄, HSO₄⁻, HCl, CH₃OH, NH₃.

It has surprisingly been found that enantiomers of etodolac and other tetra-hydropyrano indole derivatives can be directly racemized effectively upon treatment with various Lewis acids and/or Brønsted acids. The racemization reaction described herein is effective on compounds of the following general structure.

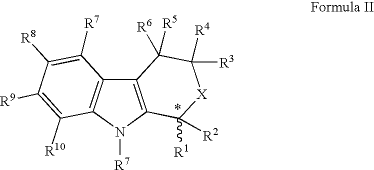

wherein R¹, R², R³, R⁴, R⁵, R⁶, R⁷, R⁸, R⁹, R¹⁰ and R¹¹ are each independently hydrogen; halogen; —CN; —NO₂; —OH; —SH; or an unsubstituted or substituted moiety selected from alkyl, alkenyl, alkynyl, alkoxy, allyloxy, aryl, heteroaryl, cycloalkyl, and heterocycloalkyl; X is O, N, or S; wherein R¹ and R² are not the same; and wherein R¹ and R² are not esters.

A preferred Lewis acid is BF₃-Et₂O and a preferred Brønsted acid is H₂SO₄. For the racemization reaction, preferred concentrations of Lewis and Brønsted acids are about 1 to about 1.5 equivalents. Preferably, the concentration is about 1.5 equivalents.

The racemization reaction is solvent and temperature dependant. Preferred solvents include 2-propanol and ethyl alcohol. Although increasing temperatures increased racemization, increasing temperatures also resulted in decomposition of the starting enantiomer. A preferred temperature range for the racemization reaction is below 50° C., preferably about 18° C. to about 40° C., more preferably about 18° C. to about 26° C. A preferred temperature for the reaction is about 20° C.

A typical racemization reaction and work up is as follows: (S)-Etodolac (0.80 g, 2.8 mmol) is dissolved in anhydrous 2-propanol (28 mL ml) under a nitrogen atmosphere, then boron trifloride diethyl etherate (0.70 mL, 5.6 mmol) is added dropwise via syringe. The resulting reaction mixture is stirred at room temperature for 18 hr. Solvent is removed under reduced pressure, and to the residue is added water (20 mL). The aqueous mixture is neutralized to pH 4-5 with saturated solution of sodium bicarbonate in water and extracted by dichloromethane (20 mL×3). The combined organic phases is dried over MgSO₄, filtered, and concentrated. The resulting residue is dried in vacuo.

A preferred racemization procedure and work up is as follows: (S)-Etodolac (0.80 g, 2.8 mmol) is dissolved in anhydrous 2-propanol (28 mL) under a nitrogen atmosphere, then boron trifloride diethyl etherate (0.70 mL, 5.6 mmol) is added dropwise via syringe. The resulting reaction mixture is stirred at room temperature for 18 hr. Solvent is removed under reduced pressure, and to the residue is added water (20 mL). The mixture is neutralized to pH 10 by addition of Na₂CO₃ and extracted with ethyl acetate (20 ml). After separation, the aqueous phase is acidified with diluted hydrochloric acid to pH 4-5 and extracted by dichloromethane (20 mL×3). The combined dichloromethane phases are dried over MgSO₄, filtered, concentrated and dried in vacuo to give 0.77 g of crude product.

### EXAMPLES

The following Examples are provided to illustrate certain aspects of the present invention and to aid those of skill in the art in practicing the invention. These Examples are in no way to be considered to limit the scope of the invention in any manner.

To identify conditions that would allow for the direct racemization of etodolac, the effects of temperature, solvent, catalyst (both Brønsted acids and Lewis acids), and the amount of catalyst on the racemization of (S)-etodolac were investigated.

### Example 1

### Effect of Temperature

In the preparation of pure (S)-etodolac via esterfication of (S)-etodolac and basic hydrolysis, significant decomposition of (S)-etodolac was observed when (S)-etodolac was refluxed in methanol. Also, faster degradation of etodolac with increasing temperature, in aqueous solution, has been reported (Lee et al., Pharm. Sci. (1988), 77, 81). Thus, varied reaction temperatures were examined for racemization of (S)-etodolac by using concentrated H₂SO₄ in 1,4-dioxane. The results, as summarized in Table 1, suggested that increasing temperature enhances the racemization, but higher temperature, in particular, over 50° C. gave rise to significant decomposition of (S)-etodolac.

TABLE 1

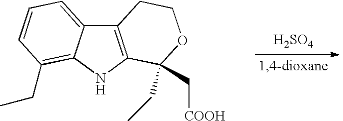

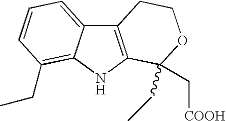

Effect of Temperature on Racemization of (S)-Etodolac^{a}

| | | results | |
|---|---|---|---|
| | temperature | | percentage of |
| entry | (° C.) | ratio of R:S of Etodolac | side products (%)^{b} |
| 1 | 20 | 0.23:1.0 | 10 |
| 2 | 50 | 0.40:1.0 | 13 |
| 3 | 100 | —^{c} | major |
| ^{a}racemization reaction conditions unless otherwise indicated: 0.37 mmol of (S)-etodolac and one equivalent of H₂SO₄, 10 ml of 1,4-dioxane, 18 h. For HPLC analysis, a portion of reaction mixture was taken out after certain hours and concentrated under reduced pressure. The residue was extracted from water with dichloromethane twice. The combined organic phases were dried over MgSO₄, filtered, and concentrated. | | | |
| # The resulting residue was dried in vacuo and then subjected to HPLC analysis by using Chiral-AGP column at 225 nm. | | | |

^{b}Ratio of peak area at 225 nm.
^{c}Thin Layer Chromatography (TLC) indicated no desired product but side products was formed.

### Example 2

### Effect of Solvent on Racemization of (S)-Etodolac

A variety of readily available organic solvents were examined for solvent effect. The data given in Table 2 indicate that 2-propanol is a good solvent, in which the racemization reaction is completed at room temperature along with the formation of a very small amount of side products.

TABLE 2

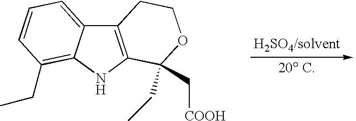

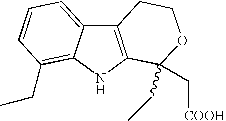

Effect of Solvent on Racemization of (S)-etodolac^{a}

| | | results | |
|---|---|---|---|
| | | | percentage of |
| entry | solvent | ratio of R:S of Etodolac | side products (%)^{b} |
| 1 | MeOH | —^{c} | —^{c} |
| 2 | CH₃CN | 0.10:1.0 | 87 |
| 3 | 1,4-dioxane | 0.22:1.0 | 11 |
| 4 | THF | no racemation | — |
| 5 | 2-propanol | 1:1 | 5 |
| ^{a}racemization reaction conditions unless otherwise indicated: 20° C., 0.37 mmol of (S)-etodolac, and 1.5 equivalent of acid in 5-10 ml of organic solvent. For HPLC analysis, a portion of reaction mixture was taken out after about 16-18 hours and concentrated under reduced pressure. The residue was extracted from water with dichloromethane twice. The combined organic phases were dried over MgSO₄, filtered, and concentrated. | | | |
| # The resulting residue was dried in vacuo and then subjected to HPLC analysis by using Chiral-AGP column at 225 nm. | | | |

^{b}Ratio of HPLC peak areas at 225 nm. In some cases, the data as to the side products are skewed because the HPLC peaks of the side products overlapped with that of either (S)-etodolac or (R)-etodolac.
^{c}TLC indicated no desired product but only methyl ester of etodolac was generated.

### Example 3

### Effects of Brønsted Acids and Lewis Acids

In order to study the effects of both Brønsted acid and Lewis acid on recemization of (S)-etodolac, concentrated H₂SO₄, HCl (as a gas solution in 1,4-dioxane), BF₃-Et₂O, SOCl₂, SnCl₄, TiCl₄, AlCl₃, and Lanthanum triflate were examined for racemization reaction of (S)-etodolac in 2-propanol. The experimental results as shown in Table 3 indicate that BF₃-Et₂O and H₂SO₄ are good acids for the racemization reaction.

TABLE 3

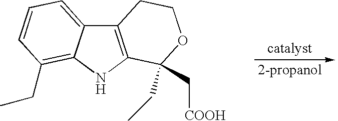

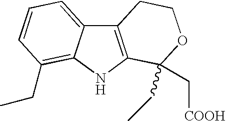

Effects of Brønsted acid and Lewis acid on
Racemization of (S)-Etodolac^{a}

| | conditions | | results | |
|---|---|---|---|---|
| | | reaction | ratio of R:S of | percentage of |
| entry | acid | time | Etodolac | side products (%)^{b} |
| 1 | SOCl₂ | 18 h | less than 0.06:1.0^{c} | less than 4^{c} |
| 2 | SnCl₄ | 18 h | 0.39:1.0 | no^{d} |
| 3 | TiCl₄ | 18 h | less than 0.06:1.0^{c} | less than 4^{c} |
| 4 | AlCl₃ | 24 h | less than 0.37:1.0^{c} | less than 27^{c} |
| 5 | La(CF₃SO₃)₃ | 24 h | less than 0.01:1.0^{c} | no^{d} |
| 6 | HCl | 21 h | 0.30:1.0 | 3 |
| 7 | BF₃-Et₂O | 15 h | 1:1 | 6% |
| 8 | H₂SO₄ | 4 h | 1:1 | 2 |
| ^{a}Racemization reaction conditions unless otherwise indicated: 20° C., 0.37 mmol of (S)-etodolac, and 1-1.5 equivalents of acid in 5 ml of organic solvent. For HPLC analysis, a portion of reaction mixture was taken out after certain hours and concentrated under reduced pressure. The residue was extracted from water with dichloromethane twice. The combined organic phases were dried over MgSO₄, filtered, and concentrated. | | | | |
| # The resulting residue was dried in vacuo and then subjected to HPLC analysis by using Chiral-AGP column at 225 nm. | | | | |

^{b}Ratio of HPLC peak areas at 225 nm. In some cases, the data are inaccurate because the HPLC peak of side products overlapped with that of either (S)-etodolac or (R)-etodolac.
^{c}The data are skewed because the peaks of side products overlapped with that of (R)-etodolac.
^{d}Not detectable by HPLC at 254 nm.

### Example 4

### Effect of the Amount of Catalyst on Racemization of (S)-Etodolac

The results given in Table 4 indicate that the use of 1.5 equivalents of BF₃-Et₂O is more practical to catalyze racemization of (S)-etodolac in 2-propanol, although the use of other equivalents of BF₃-Et₂O can be used for the racemization reaction.

TABLE 4

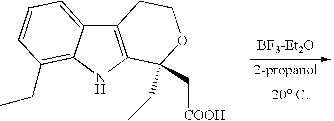

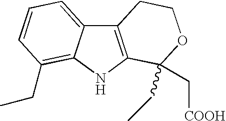

Effect of the Amount of Catalyst on Racemization of (S)-etodolac^{a}

| | | | results | |
|---|---|---|---|---|
| | equivalent of | | | percentage of |
| | BF₃-Et₂O | reaction time | ratio of R:S of | side products |
| entry | to (S)-etodolac | (h) | Etodolac | (%)^{b} |
| 1 | 0.75 | 23 | less than 0.15:1.0 | —^{c} |
| 2 | 1.0 | 15 | 0.88:1.0 | 7 |
| 3 | 1.5 | 5 | 0.33:1.0 | no^{d} |
| 4 | | 15 | 1:1 | less than 3 |
| ^{a}Racemization reaction conditions unless otherwise indicated: 20° C., 0.37 mmol of (S)-etodolac, and 5.0 ml of dry 2-propanol. For HPLC analysis, a portion of reaction mixture was taken out after about certain hours and concentrated under reduced pressure. The residue was extracted from water with dichloromethane twice. The combined organic phases were dried over MgSO₄, filtered, and concentrated. | | | | |
| # The resulting residue was dried in vacuo and then subjected to HPLC analysis by using Chiral-AGP column at 225 nm. | | | | |

^{b}Ratio of HPLC peak areas at 225 nm. In some cases, the data are skewed because the HPLC peak of side products overlapped with that of either (S)-etodolac or (R)-etodolac.
^{d}Not detectable by HPLC at 254 nm.

### Example 5

### Racemization of (R)-Etodolac

The above methods for racemization of (S)-etodolac can also be applied to (R)-etodolac. The data listed in Table 5 indicate that both BF₃-Et₂O and concentrated H₂SO₄ are able to racemize (R)-etodolac efficiently.

TABLE 5

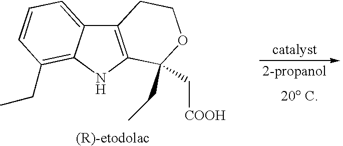

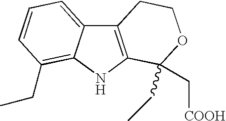

Racemization of (R)-etodolac by BF₃-Et₂O and concentrated H₂SO₄^{a}

| | | | results | |
|---|---|---|---|---|
| | | reaction time | ratio of R:S of | percentage of |
| entry | acid | (h) | Etodolac | side products (%)^{b} |
| 1^{c} | BF₃-Et₂O | 22 | 1.3:1.0 | 1 |
| 2^{d} | H₂SO₄ | 23 | 1.0:1.0 | 5 |
| ^{a}Racemization reaction conditions unless otherwise indicated: 20° C., 0.37 mmol of (R)-etodolac, and 5.0 ml of dry 2-propanol. For HPLC analysis, a portion of reaction mixture was taken out after about 16-18 hours and concentrated under reduced pressure. The residue was extracted from water with dichloromethane twice. The combined organic phases were dried over MgSO₄, filtered, and concentrated. | | | | |
| # The resulting residue was dried in vacuo and then subjected to HPLC analysis by using Chiral-AGP column at 225 nm. | | | | |

^{b}Ratio of HPLC peak areas at 225 nm.
^{c}1.5 equivalents of BF₃-Et₂O.
^{d}One equivalent of H₂SO₄.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the spirit and scope of the invention. More specifically, it will be apparent that certain solvents which are both chemically and physiologically related to the solvents disclosed herein may be substituted for the solvents described herein while the same or similar results would be achieved. All such similar substitutes and modifications apparent to those skilled in the art are deemed to be within the spirit and scope of the invention as defined by the appended claims.

All patents, patent applications, and publications mentioned in the specification are indicative of the levels of those of ordinary skill in the art to which the invention pertains. All patents, patent applications, and publications are herein incorporated by reference to the same extent as if each individual publication was specifically and individually indicated to be incorporated by reference.

The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A process for the racemization of either the R-enantiomer or S-enantiomer of Formula II 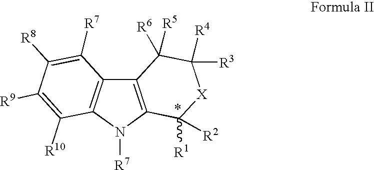
wherein R¹, R², R³, R⁴, R⁵, R⁶, R⁷, R⁸, R⁹, R¹⁰ and R¹¹ are each independently hydrogen;
halogen; —CN; —NO₂; —OH; —SH; or an unsubstituted or substituted moiety selected from alkyl, alkenyl, alkynyl, alkoxy, allyloxy, aryl, heteroaryl, cycloalkyl, and heterocycloalkyl; X is O, N, or S; wherein R¹ and R² are not the same; wherein R¹ and R² are not esters; and wherein said process comprises:
a) adding a compound of Formula II to a reaction solvent; and
b) adding a Lewis acid and/or Brønsted acid to the reaction mixture; and
wherein if the acid is H₂SO₄, HCl, or para-toluensulfonic acid the reaction solvent is not methyl alcohol.

2. The process according to claim 1 further comprising:
removing the reaction solvent after the reaction is complete; and
removing the Lewis acid and/or Brønsted acid after the reaction is complete.

3. The process according to claim 1, wherein the reaction solvent is a lower alcohol, CH₃CN, 1,4-dioxane, or CH₂Cl₂.

4. The process of claim 3, wherein the reaction solvent is selected from one or more of the group consisting of methyl alcohol, 2-methyl propan-1-ol, butan-2-ol, 2-propanol, ethyl alcohol and tert-butyl alcohol.

5. The process of claim 4, wherein the reaction solvent is 2-propanol.

6. The process of claim 4, wherein the reaction solvent is ethyl alcohol.

7. The process of claim 1, wherein the Lewis acid and/or Brønsted acid is selected from the group consisting of one or more of SOCl₂, SnCl₄, TiCl₄, AlCl₃, La(CF₃SO₃)₃, BF₃-Et₂O and H₂SO₄.

8. The process of claim 7, wherein the acid is BF₃-Et₂O.

9. The process of claim 7, wherein the acid is H₂SO₄.

10. The process of claim 1 further comprising continuing the reaction for about 30 minutes to about 36 hours.

11. The process of claim 10, wherein the reaction is continued for about 4 to about 20 hours.

12. The process of claim 1, wherein the reaction is carried out at a temperature below 50° C.

13. The process according to claim 12, wherein the reaction is carried out at about 18° C. to about 26° C.

14. The process according to claim 13, wherein the reaction is carried out at about 20° C.

15. The process according to claim 1, wherein the concentration of the Lewis and/or Brønsted acid is about 0.75 to about 1.5 equivalents.

16. The process according to claim 15, wherein the concentration of acid is about 1.5 equivalents.

17. The process according to claim 1, wherein the compound of Formula II is etodolac.

18. The process according to claim 1, wherein the R- or S-enantiomer of Formula II is selected from the group consisting of

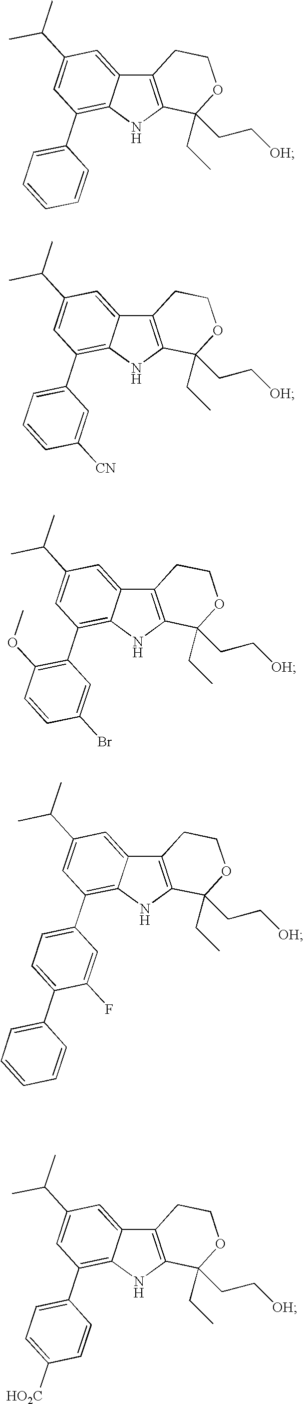

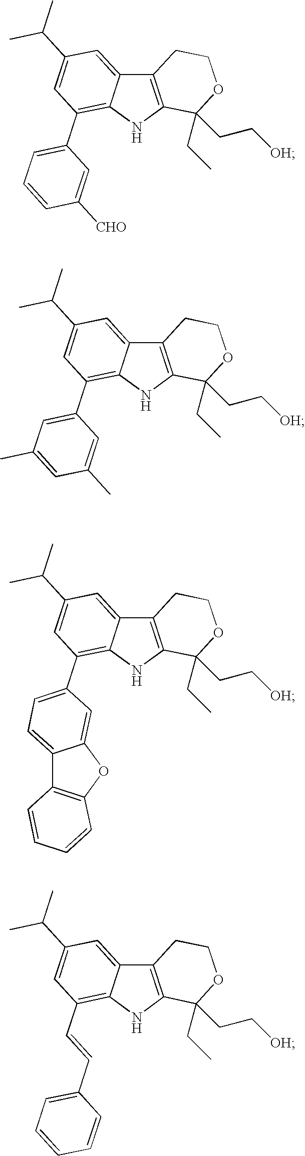

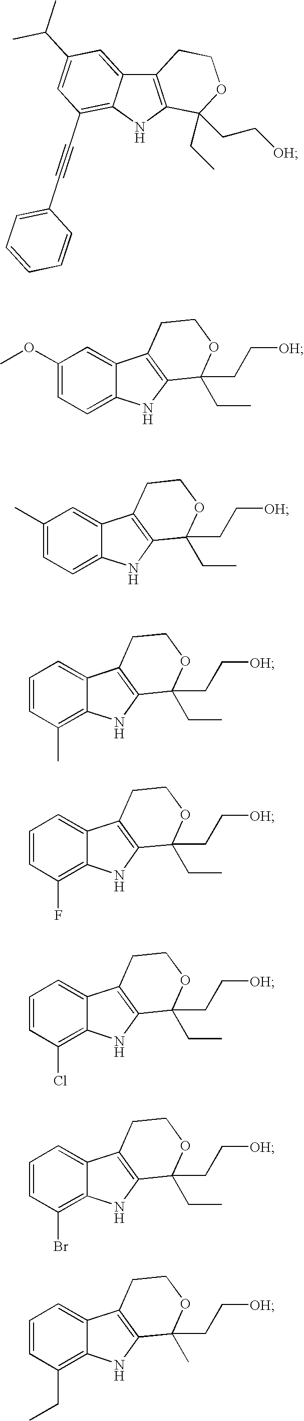

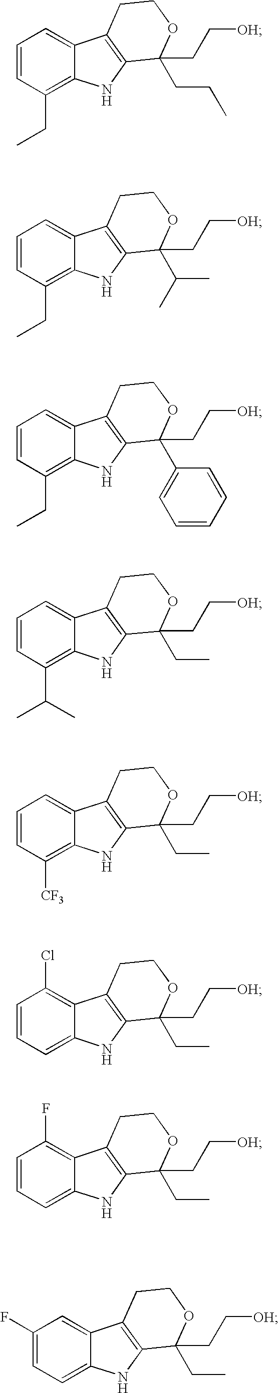

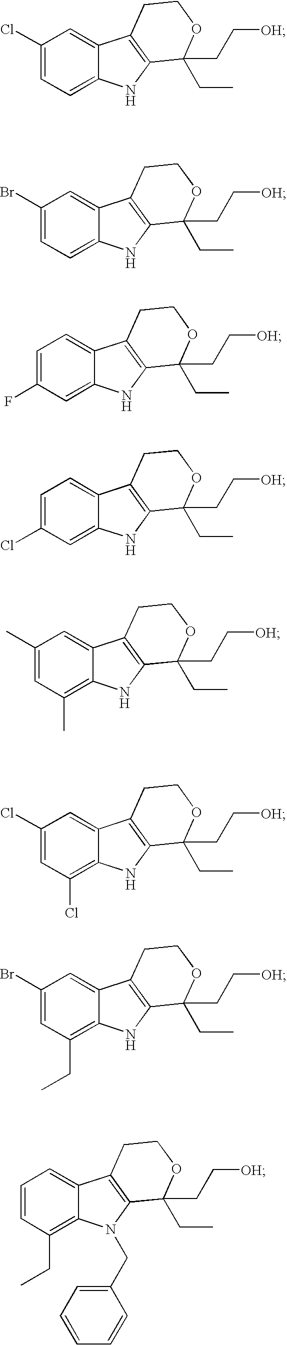

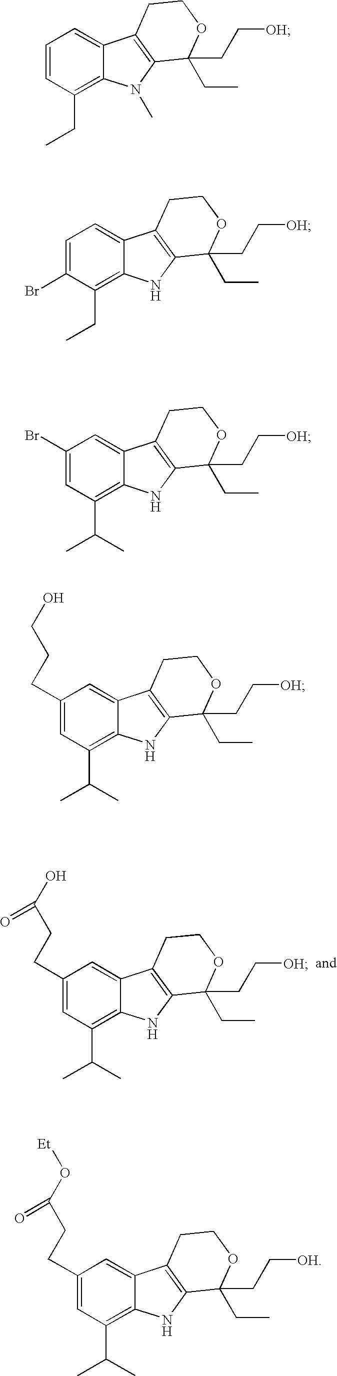

19. The process according to claim 18, wherein the R- or S-enantiomer of Formula II has the following substituents: X is O; R¹ is —CH₂CH₃; R² is —CH₂CH₂OH; R⁸ is Br; R¹⁰ is —CH₂CH₃; and R³, R⁴, R⁵, R⁶, R⁷, R⁹, and R¹¹ are each H.

20. The process according to claim 18, wherein the R- or S-enantiomer of Formula II has the following substituents: X is O; R¹ is —CH₂CH₃; R² is —CH₂CH₂OH; R¹⁰ is —CH(CH₃)₂; and R³, R⁴, R⁵, R⁶, R⁷, R⁸, R⁹, and R¹¹ are each H.

21. The process according to claim 18, wherein the R- or S-enantiomer of Formula II has the following substituents: X is O; R¹ is —CH₂CH₃; R² is —CH₂CH₂OH; R⁸ is —CH₂CH₂C(═O)OCH₂CH₃; R¹⁰ is —CH(CH₃)₂; and R³, R⁴, R⁵, R⁶, R⁷, R⁹, and R¹¹ are each H.

22. The process according to claim 18, wherein the R- or S-enantiomer of Formula II has the following substituents: X is O; R¹ is —CH₂CH₃; R² is —CH₂CH₂OH; R⁸ is Br; R¹⁰ is —CH(CH₃)₂; and R³, R⁴, R⁵, R⁶, R⁷, R⁹, and R¹¹ are each H.

23. A process for the racemization of either the R-enantiomer or S-enantiomer of Formula II, as defined in claim 1, comprising:
a) adding the compound to be racemized to 2-propanol; and
b) adding a Lewis acid and/or Brønsted acid to the reaction mixture.

24. A process according to claim 23 further comprising running the reaction at a temperature under 50° C.

25. A process according to claim 24, wherein the reaction is run at about 18° C. to about 26° C.

26. A process according to claim 24, wherein the reaction is run at about 20° C.

27. A process according to claim 23, wherein the Lewis acid and/or Brønsted acid is selected from the group consisting of one or more of the group consisting of SOCl₂, SnCl₄, TiCl₄, AlCl₃, La(CF₃SO₃)₃, BF₃-Et₂O and H₂SO₄.

28. A process according to claim 27, wherein the acid is BF₃-Et₂O.

29. A process according to claim 27, wherein the acid is H₂SO₄.

30. A process according to claim 23, wherein the acid is added at a concentration of about 0.75 to about 1.5 equivalents.

31. A process according to claim 23 further comprising: c) removing the 2-propanol to form a residue; adding water to the residue after the removal of the 2-propanol to form an aqueous mixture; neutralizing the aqueous mixture to pH 4-5; extracting the aqueous mixture with dichloromethane to form one or more organic phases; drying the organic phases over MgSO₄; filtering the organic phases; concentrating the organic phases; and drying in vacuo.

32. A process according to claim 23 further comprising continuing the reaction for at least about 30 minutes to about 36 hours.

33. A process according to claim 23, wherein the compound of Formula II is etodolac.

34. A process for the racemization of either the R-enantiomer or S-enantiomer of Formula II, as defined in claim 1, comprising:
a) adding the compound to be racemized to anhydrous 2-propanol; and
b) adding boron trifloride diethyl etherate.

35. A process according to claim 34, further comprising continuing the reaction for at least about 30 minutes to about 36 hours.

36. A process according to claim 34, wherein the reaction is continued for about 4 to about 20 hours.

37. A process according to claim 34 further comprising running the reaction at a temperature below 50° C.

38. A process according to claim 37, wherein the reaction is carried out at about 18° C. to about 26° C.

39. A process according to claim 34, wherein the boron trifluoride is added at a concentration of about 0.75 to about 1.5 equivalents.

40. A process according to claim 34 further comprising adding the compound to be racemized under a nitrogen atmosphere.

41. A process according to claim 34 further comprising running the reaction for about 30 min to 34 hours.

42. A process according to claim 34, further comprising: c) removing the 2-propanol under reduced pressure; adding water to the resulting residue to form an aqueous mixture;
neutralizing the aqueous mixture to about pH 10; extracting the aqueous mixture with ethyl acetate; acidifying the aqueous mixture to about pH 4-5; extracting the aqueous mixture with dichloromethane to form one or more organic phases; drying the organic phases over MgSO₄; filtering and concentrating the dried organic phases; and drying in vacuo.

43. A process according to claim 34, wherein the compound of Formula II is etodolac.

44. A process according to claim 34, wherein the R- or S-enantiomer of Formula II is selected from the group consisting of 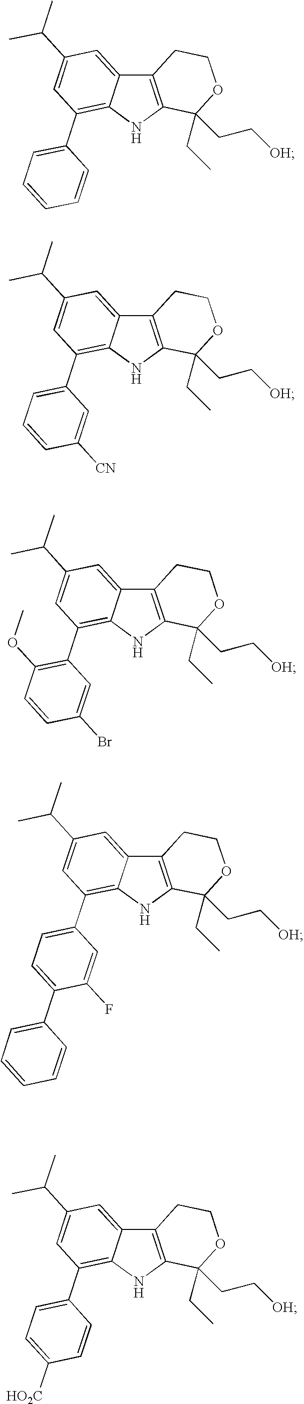 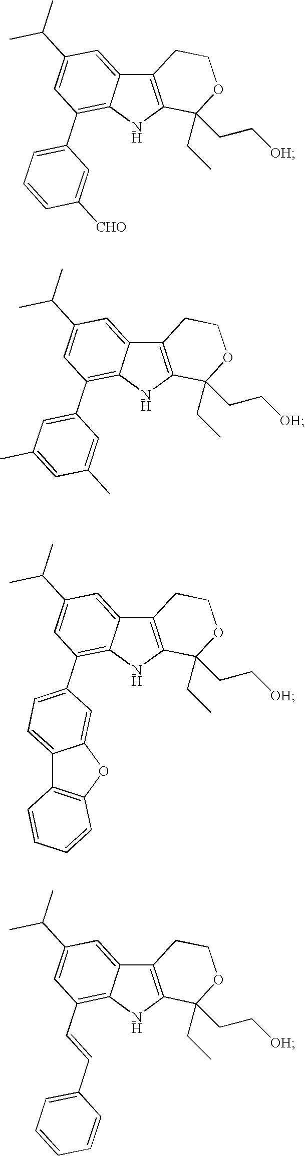 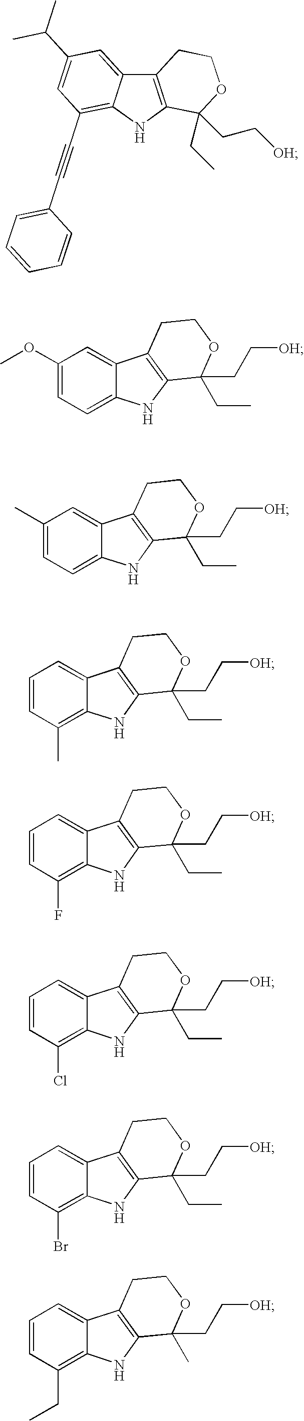 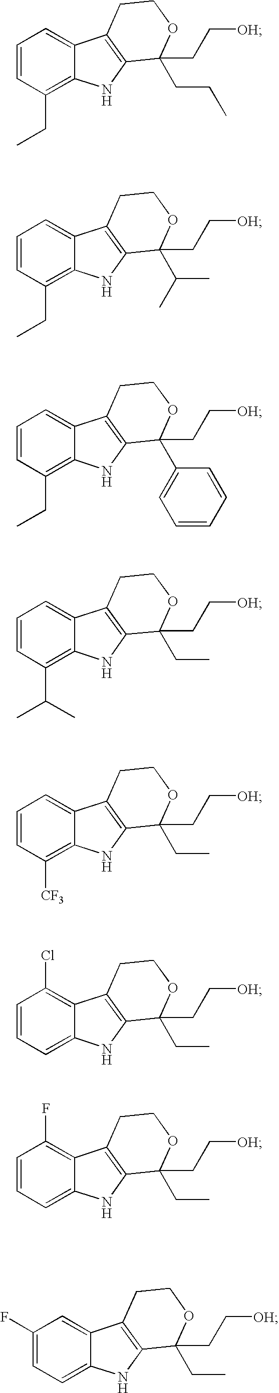 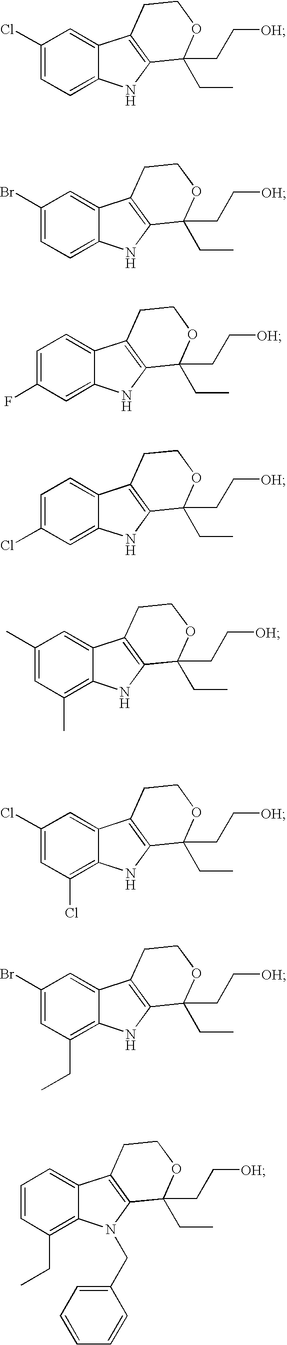 [chemical structure]
